(19) United States
(12) Patent Application Publication
(10) Pub. No.: US 2021/0295999 A1
(43) Pub. Date: Sep. 23, 2021

(54) PATIENT STATE PREDICTION APPARATUS, PATIENT STATE PREDICTION METHOD, AND PATIENT STATE PREDICTION PROGRAM

(71) Applicant: Hitachi, Ltd., Tokyo (JP)

(72) Inventors: Zisheng LI, Tokyo (JP); Masahiro OGINO, Tokyo (JP); Kitaro YOSHIMITSU, Tokyo (JP); Yoshiki UCHIO, Tokyo (JP)

(21) Appl. No.: 17/078,206

(22) Filed: Oct. 23, 2020

(30) Foreign Application Priority Data
Mar. 18, 2020 (JP) 2020-047824

(51) Int. Cl. G16H 50/30; G16H 10/60; G16H 50/20; G16H 50/70; G16H 70/20; G16H 70/60; G16H 15/00

(52) U.S. Cl. CPC G16H 50/30; G16H 10/60; G16H 50/20; G16H 15/00; G16H 70/20; G16H 70/60; G16H 50/70

(57) ABSTRACT

A prediction method to predict a patient state with high accuracy by generating a fused feature quantity that can reflect a feature quantity of a large number of types of data includes an analysis step of extracting feature quantities by analyzing biometric information of a patient and medical care information of the patient other than the biometric information, a fusing step of generating a fused feature quantity by fusing the biometric information feature quantity and the medical care information feature quantity, a learning step of learning a relationship between the biometric information feature quantity and the medical care information feature quantity, a feature quantity mutation learning step of predicting the fused feature quantity by the feature quantity relationship learning from the input of only the biometric information, and a prediction step of predicting a patient state by using the predicted fused feature quantity obtained by the feature quantity mutation learning.

### INCORPORATION BY REFERENCE

The present application claims priority from Japanese patent application JP-2020-047824 filed on Mar. 18, 2020, the content of which is hereby incorporated by reference into this application.

### BACKGROUND OF THE INVENTION

### Field of the Invention

The invention relates to a technique for predicting a condition change or an abnormality of a patient.

### Description of the Related Art

An intensive care unit (ICU) monitors vital data (biometric information) regarding respiration, circulation, centrum, infection, renal function, blood, and the like of a patient and performs triage of the patient. However, with the increase in the number of patients and the shortage of manpower for doctors and nurses specializing in the ICU, reducing the load on site is an urgent problem.

Recent years, in order to solve the problem, research and development in predicting a condition change or a sign of aggravation of a patient by using biometric information and medical care information of the patient and applying a prediction result to a doctor's diagnosis support have been carried out. US 2018/0,025,290 A discloses a prediction technique using machine learning. Specifically, a technique for generating learning parameters of a rule-based linear model by using a large number of pieces of biometric information and applying the learning parameters to predict a patient risk for an actual patient is described.

In a general prediction technique using machine learning, it is necessary to match a type of learning data for generating a learning model with a type of input data for predicting (inferring) during operation. In addition, in order to obtain higher prediction accuracy, it is preferable to abundantly utilize medical care information such as medical care records and test results of patients, which are considered to greatly contribute to the prediction of the patient state, and to use the medical care information for the learning and the inference. However, in various hospitals and facilities that operate the ICU, there is a high possibility that the type and number of pieces of the biometric information and the medical care information of the patient that are recorded differ depending on the facility. For this reason, the learning data used at the time of learning the prediction model is generally limited to the patient biometric information and the patient medical care information that are commonly recorded in many facilities and can be input to the prediction system. For example, in US 2018/0,025,290 A, as the premise, the biometric information for the learning and the biometric information for inference have the same number of types in order to apply the generated learning parameter to the inference and to obtain a desired prediction result. Furthermore, the medical care information of the patient is not used.

### SUMMARY OF THE INVENTION

The invention is to provide a prediction method and a prediction apparatus that can predict patient risk with high accuracy similarly to when using a larger number of types and a larger number of pieces of patient information by inputting patient information obtained from facility even though the patient information obtained from each facility is different.

In order to achieve the above-mentioned object, in the invention, in the prediction of a patient state, a learning model is generated by learning a relationship between a feature quantity obtained by fusing a large number of pieces of patient biometric information and patient medical care information or a feature quantity obtained by integrating a large number of items of biometric information and individual patient biometric information, and the learning model is incorporated into a prediction model that predicts patient risk. This prediction model predicts and outputs a patient risk by inputting patient information of which the type and number of pieces are not matched with those of the learning data of the learning model.

Specifically, a prediction apparatus according to the invention includes: a feature extraction unit that receives biometric information of a patient as an input and extracts a feature quantity of the biometric information; a feature mutation unit that receives the feature quantity of the biometric information extracted by the feature extraction unit as an input and outputs a predicted value of an integrated feature quantity of patient information including biometric information other than the biometric information input to the feature extraction unit; and a state prediction unit that receives the predicted value of the integrated feature quantity as an input and outputs risk information of the patient. The integrated feature quantity is a feature quantity obtained by integrating the feature quantities extracted from the patient information including a large number of pieces of biometric information and is generated for each patient information. The feature mutation unit is a unit in which the relationship is learned by using the created integrated feature quantity and the feature quantities of individual biometric information.

In addition, a prediction method according to the invention includes: an analysis step of analyzing patient information including a large number of pieces of biometric information and extracting a feature quantity of the patient information for each type of the patient information or each item of the biometric information; an integration step of generating an integrated feature quantity (fused feature quantity) by integrating the feature quantities extracted for each type or each item; a learning step of learning a relationship between the feature quantity for each type or each item and the integrated feature quantity; a feature quantity mutation step of predicting a predicted value of the integrated feature quantity based on the relationship obtained in the learning step, by using a feature quantity of a specific biometric information as an input; and a prediction step of predicting a specific state of a patient by using the predicted value of the integrated feature quantity.

According to the invention, when operating in individual facilities, even though the type and number of pieces of patient information (biometric information and medical care information) input to the prediction model is not matched with the type and number of pieces of learning data, it is possible to perform inference similar to the inference based on a large number of types of the biometric information and the medical care information, and it is possible to predict a patient state with high accuracy.

### BRIEF DESCRIPTION OF THE DRAWINGS

FIG. 1 is a block diagram illustrating an example of a configuration of a prediction apparatus of the invention;

FIG. 2 is a block diagram illustrating an example of a hardware configuration of a prediction apparatus according to a first embodiment;

FIGS. 3A and 3B are diagrams illustrating an outline of processing of a prediction method according to the first embodiment, in which FIG. 3A is a diagram illustrating processing at the time of learning and FIG. 3B is a diagram illustrating processing at the time of inference;

FIG. 4 is a flowchart illustrating a processing flow at the time of learning of a prediction model of the first embodiment;

FIGS. 5A to 5C are diagrams illustrating a configuration of a biometric information analysis unit (feature extraction unit) of the first embodiment, in which FIGS. 5A to 5C illustrate different configuration examples;

FIG. 6 is a diagram illustrating a configuration of a medical care information analysis unit (feature extraction unit) of the first embodiment;

FIG. 7 is a diagram illustrating a configuration related to learning in a feature quantity processing unit of the first embodiment;

FIG. 8 is a diagram illustrating a configuration of a state prediction unit of the first embodiment;

FIG. 9 is a flowchart illustrating a processing flow at the time of inference of a prediction model of the first embodiment;

FIG. 10 is a diagram illustrating a configuration related to inference in a feature quantity processing unit of the first embodiment;

FIGS. 11A to 11C are diagrams illustrating examples of output of a prediction result of the prediction apparatus according to the first embodiment;

FIGS. 12A and 12B are diagrams illustrating an outline of processing of a prediction method according to a second embodiment, in which FIG. 12A is a diagram illustrating processing at the time of learning and FIG. 12B is a diagram illustrating processing at the time of inference;

FIG. 13 is a flowchart illustrating a processing flow at the time of learning of a prediction model of the second embodiment;

FIG. 14 is a diagram illustrating a configuration related to learning in a feature mutation unit of the second embodiment;

FIG. 15 is a flowchart illustrating a processing flow at the time of inference of a prediction model according to the second embodiment; and

FIG. 16 is a diagram illustrating a configuration related to inference in the feature mutation unit of the second embodiment.

### DETAILED DESCRIPTION OF THE INVENTION

Hereinafter, embodiments of the invention will be described in detail with reference to the drawings. In addition, in all the drawings for explaining the embodiments, in principle, the same components are denoted by the same reference numerals, and redundant description thereof will be omitted.

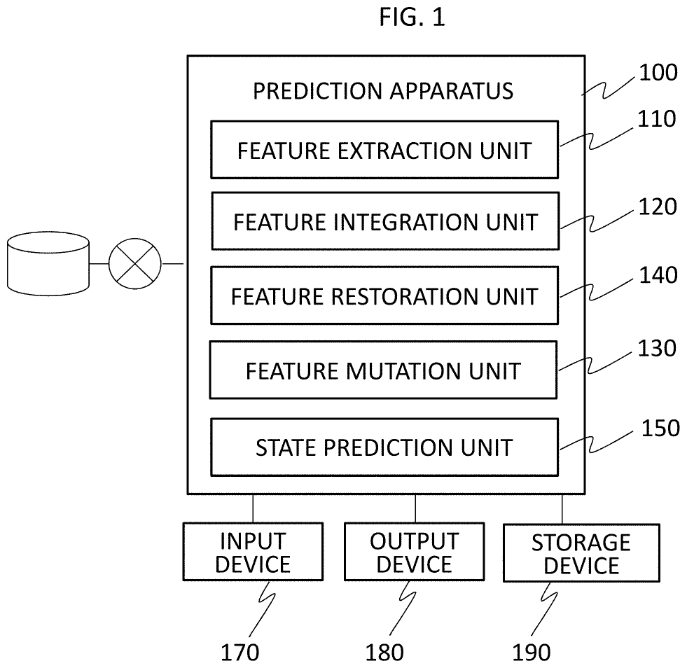

First, the overall configuration of a prediction apparatus common to each embodiment will be described with reference to FIG. 1.

As illustrated in FIG. 1, a prediction apparatus 100 includes a feature extraction unit 110 that receives biometric information of a patient as an input and extracts a feature quantity of the biometric information, a feature mutation unit 130 that receives the feature quantity of the biometric information extracted by the feature extraction unit 110 as an input and outputs a predicted value of an integrated feature quantity of patient information including a large number of pieces of the biometric information other than the biometric information input to the feature extraction unit 110, and a state prediction unit 150 that receives the predicted value of the integrated feature quantity as an input and outputs risk information of the patient.

The feature extraction unit 110, the feature mutation unit 130, and the state prediction unit 150 constituting the prediction apparatus 100 can be partially or entirely configured with a learning model. The learning model is learned so that a predetermined output is obtained for a predetermined input.

The prediction apparatus 100 may further include a feature integration unit 120 that receives the feature quantity extracted for each of the patient information including a large number of pieces of the biometric information as the learning data of the feature mutation unit 130 as an input and generates the integrated feature quantity. The feature quantity of the patient information input to the feature integration unit 120 can also be extracted by the feature extraction unit 110. In addition, a feature restoration unit 140 that restores the feature quantity of the patient information from the integrated feature quantity may be provided at the subsequent stage of the feature integration unit 120, and thus, a learning effect of the learning model can be checked from the feature quantity restored by the feature restoration unit 140. The feature integration unit 120 and the feature restoration unit 140 can also be constructed by a learning model including an encoder and a decoder.

The prediction apparatus 100 according to this embodiment may further include, for example, an input device 170 for inputting data to the feature extraction unit 110 and a command for each processing, an output device 180 for outputting prediction information of the state prediction unit 150, a storage device 190 for storing data during the processing, processing results, and the like. In addition, the storage device 190 may store learning data for learning of the prediction apparatus 100 described later and the like. Furthermore, the prediction apparatus 100 may be connected to a database in a medical institution via a communication unit and the like.

Hereinafter, the outline of a function of each unit will be described.

The feature extraction unit 110 acquires the biometric information and the medical care information directly from a biometric information measurement device (not illustrated) or via the input device 170 or via the storage device 190 (including a database) and extracts the feature quantities. The biometric information is time-series measurement information obtained by measurement, such as blood pressure, heartbeat, oxygen saturation, respiration rate, body temperature, and the like. The medical care information is information obtained at the time of medical care, such as sex, age, weight, habit (smoking, drinking), and the like. Herein, the biometric information and the medical care information are collectively referred to as patient information. The extraction of the feature quantity may be, for example, extraction (rule-based extraction) of a statistical numerical value such as an average value or a maximum value of the time-series data or may be extraction by using a feature extraction algorithm or machine learning (a neural network: including DNN). The feature quantity can be generated in the form of, for example, a high-dimensional vector.

In the learning step of the prediction apparatus 100, the feature extraction unit 110 extracts the feature quantities for a large number of pieces of the patient information. As the large number of pieces of the patient information, a large number of different types of the patient information such as the biometric information and the medical care information of the patient may be used, or a large number of items of the same type of the patient information (biometric information) may be used. In a case where the biometric information and the medical care information are input, the feature quantity is extracted for each information. In addition, in a case where the large number of items of biometric information are input, the feature quantity for each item is extracted.

The feature integration unit 120 receives the feature quantities of a large number of pieces of the patient information as an input and integrates the feature quantities to create an integrated feature quantity. As the integrated feature quantity, a large number of the integrated feature quantities are obtained according to the type and item of the input patient information. The individual integrated feature quantity is obtained by converting the feature quantities of which number of types is large or of which number of pieces is large into a single or low-dimensional feature quantity that is easy to handle. For example, in a case where the feature quantities of two or more pieces of the patient information are the feature quantities of the different types of the patient information (for example, in the case of the respective feature quantities of the biometric information and the medical care information), the individual integrated feature quantity may be a fused feature quantity obtained by coupling the feature quantities, or in the case of a large number of feature quantities, the individual integrated feature quantity may be a feature quantity (compressed feature quantity) obtained by compressing a feature quantity configured with a multi-dimensional vector.

In the learning step, the feature mutation unit 130 performs learning of associating a large number of the integrated feature quantities generated by the feature integration unit 120 with the feature quantities of individual biometric information. Therefore, at the inference step, one piece of the biometric information is input, and the integrated feature quantity associated with the biometric information is output. The function of the feature mutation unit 130 can be realized by, for example, a learning model (a set of an encoder and a decoder each configured with the neural network) according to an auto-encoder method.

The feature restoration unit 140 can be provided in the subsequent stage of the feature integration unit 120 and outputs the learning result of the feature mutation unit and the restored patient information. This output can be displayed for the checking of the learning effect and the checking of the processing result at the time of inference.

The state prediction unit 150 receives the integrated feature quantity which is the output of the feature mutation unit 130 as an input and outputs patient risk information. The state prediction unit 150 is also configured by a machine learning algorithm such as a neural network NN (including DNN), and thus, a prediction model constituting the state prediction unit 150 can be obtained by learning a large number of integrated feature quantities and the patient risk information obtained at the time of learning of the feature mutation unit 130 as learning data.

The patient risk information includes, for example, information calculated in the form of a score obtained by weighting and adding a plurality of measurement items of the biometric information and text information that is further converted from the afore-mentioned information. The prediction result of the state prediction unit 150 is displayed on the output device 180.

In the prediction apparatus 100 having the above-described configuration, in the learning step, learning is performed so that the feature extraction unit 110 extracts each feature quantity by using a large number of pieces of the patient information, the feature integration unit 120 creates the integrated feature quantity by using the feature quantities, and the feature mutation unit 130 associates the biometric information feature quantity extracted from the biometric information with the integrated feature quantity created by the feature integration unit 120 to output the integrated feature quantity (predicted value) corresponding to the specific biometric information. The state prediction unit 150 performs learning so as to receive the integrated feature quantity as an input and to output the patient risk.

In the inference step, when the biometric information obtained from the patient who is a prediction target is input, the feature extraction unit 110 extracts the feature quantity of the input biometric information, and the feature mutation unit 130 outputs the predicted value of the integrated feature quantity from the extracted feature quantity. The state prediction unit 150 receives the predicted value which is the output of the feature mutation unit 130 as an input and outputs the risk information of the patient. Therefore, it is possible to perform the prediction of the patient risk.

According to the prediction apparatus according to this embodiment, even though available patient information is different for each facility, it is possible to predict the condition change and the like of the patient by inputting only the biometric information obtained from the patient information. Since the prediction is performed not only based on the specific biometric information but also the patient information that is important for determining the patient state, the prediction is performed with high accuracy.

Based on the configuration of the embodiment of the prediction apparatus described above, the details of each unit of the prediction apparatus and the embodiment of the prediction method will be described below.

### First Embodiment

In this embodiment, performed is a prediction method including: an analysis step of extracting feature quantities by analyzing biometric information of a patient and medical care information of the patient other than the biometric information, a fusing step of generating a fused feature quantity by fusing the biometric information feature quantity and the medical care information feature quantity, a learning step of learning a relationship between the biometric information feature quantity and the medical care information feature quantity, a feature quantity mutation learning step of predicting the fused feature quantity by the feature quantity relationship learning from the input of only the biometric information, and a prediction step of predicting a patient state by using the predicted fused feature quantity obtained by the feature quantity mutation learning.

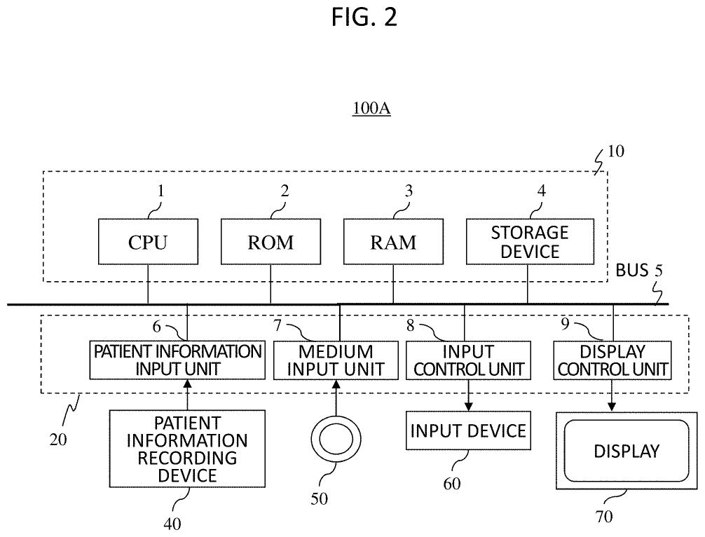

Hereinafter, a specific configuration example of a prediction apparatus 100A according to the first embodiment will be described in detail. FIG. 2 illustrates an example of the overall configuration of the prediction apparatus 100A. The prediction apparatus 100A is mainly divided into a calculation unit 10 and a user interface (UI) unit 20, and the calculation unit 10 and the UI unit 20 are connected to each other via a bus 5.

The calculation unit 10 includes a CPU (processor) 1 and memories 2 and 3. The memory includes, for example, a ROM (nonvolatile memory: read-only storage medium) 2 and a RAM (volatile memory: data readable/writable storage medium) 3. In addition, an external storage device 4 may be connected.

The UI unit 20 has an input unit for receiving data and commands from the outside and an output unit for outputting results of the prediction apparatus 100A, GUI, and the like. The input unit includes, for example, a patient information input unit 6 that is connected to a patient information recording device 40 of a medical institution or the like, a medium input unit 7 that receives an input from a recording medium 50 such as a USB, and an input control unit 8 that receives an input from an input device 60 such as a mouse or a keyboard. In the illustrated example, a display control unit 9 is provided as an output unit, and the display control unit 9 is connected to a display 70.

At least one of the memories (the ROM 2 and the RAM 3) stores in advance programs, data, and a prediction model required for realizing operations of the prediction apparatus 100A by the calculation processing of the CPU 1. The CPU 1 executes programs stored in the memory in advance to realize various processing of the prediction apparatus 100A described in detail later. In addition, the program executed by the CPU 1 may be stored in the recording medium 50 such as an optical disk, and the medium input unit 7 such as an optical disk drive may read the program and store the program in the RAM 3. In addition, the program may be stored in the storage device 4, and the program may be loaded from the storage device 4 into the RAM 3. In addition, the program may be stored in the ROM 2 in advance.

The patient information input unit 6 is an interface for acquiring the biometric information and medical care information of the patient stored in the patient information recording device 40. The storage device 4 is a magnetic storage device that stores the biometric information of the patient and the like input via the patient information input unit 6. The storage device 4 may include, for example, a nonvolatile semiconductor storage medium such as a flash memory. In addition, the external storage device connected via a network or the like may be used.

The input device 60 is a device that receives a user operation and includes, for example, a keyboard, a trackball, an operation panel, and the like. The input control unit 8 is an interface that receives an operation input that is input by a user. The operation input received by the input control unit 8 is processed by the CPU 1. The display control unit 9 performs control of allowing, for example, the patient information and the prediction result obtained by the processing of the CPU 1 to be displayed on the display 70. The display 70 displays the patient information and the prediction result under the control of the display control unit 9.

The prediction apparatus 100A according to this embodiment performs the prediction of the patient state from the biometric information by using the prediction model installed in the CPU 1. In order to realize the prediction function based on the prediction model, first, the learning of the machine learning model constituting the prediction apparatus 100A is performed, and prediction (inference) using the learned model, that is, the prediction model is performed.

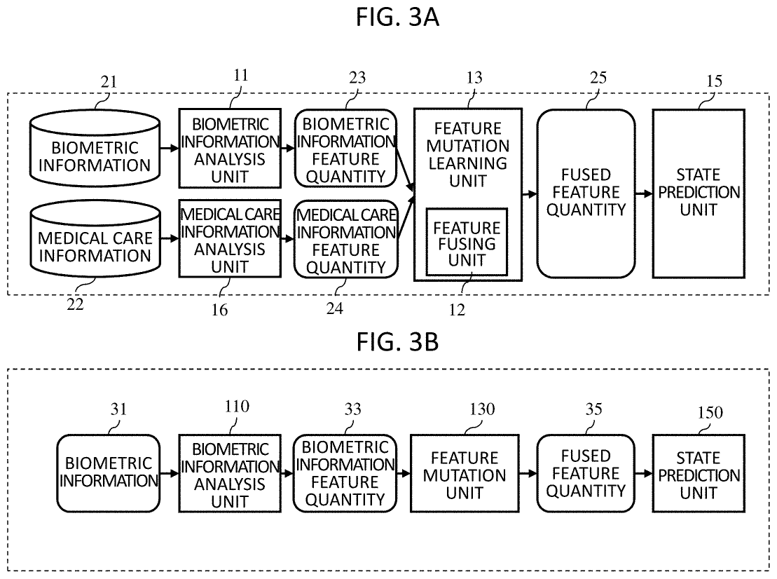

The outline of two functions for operating the prediction apparatus 100A, that is, a learning function for generating a prediction model and a prediction function using the prediction model are illustrated in the block diagrams of FIGS. 3A and 3B, respectively. As illustrated in FIG. 3A, as functions for generating a prediction model, a biometric information analysis unit (feature extraction unit) 11, a medical care information analysis unit 16, a feature mutation learning unit 13, and a state prediction unit 15 are provided to the CPU 1. The feature mutation learning unit 13 includes a feature fusing unit 12 that fuses the feature quantities generated by the biometric information analysis unit 11 and the medical care information analysis unit 16 to generate a fused feature quantity. Each of the units is basically configured by a machine learning model, and the biometric information analysis unit 110, the feature mutation unit 130, and the state prediction unit 150 illustrated in FIG. 3B are generated by performing predetermined learning. Then, the generated biometric information analysis unit (feature extraction unit) 110, the generated feature mutation unit 130, and the generated state prediction unit 150 perform the prediction of a predetermined patient state.

[Learning of Learning Model]

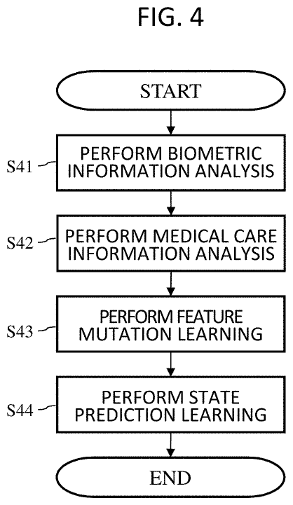

First, the processing of a learning function block illustrated in FIG. 3A will be described with reference to the learning function flowchart illustrated in FIG. 4.

[Step S41]

The biometric information analysis unit 11 receives biometric information 21, which is vital data of the patient, and extracts a biometric information feature quantity 23 by using a machine learning or rule-based method.

The biometric information is a time-series measurement signal such as blood pressure, heartbeat, oxygen saturation, respiration rate, and body temperature, and in the learning step, measurement signals acquired in advance for a large number of patients are used. The biometric information analysis unit 11 extracts a feature quantity capable of representing a temporal change in the biometric information from the measurement signals by using a long short-term memory (LSTM) method, which is a known machine learning method.

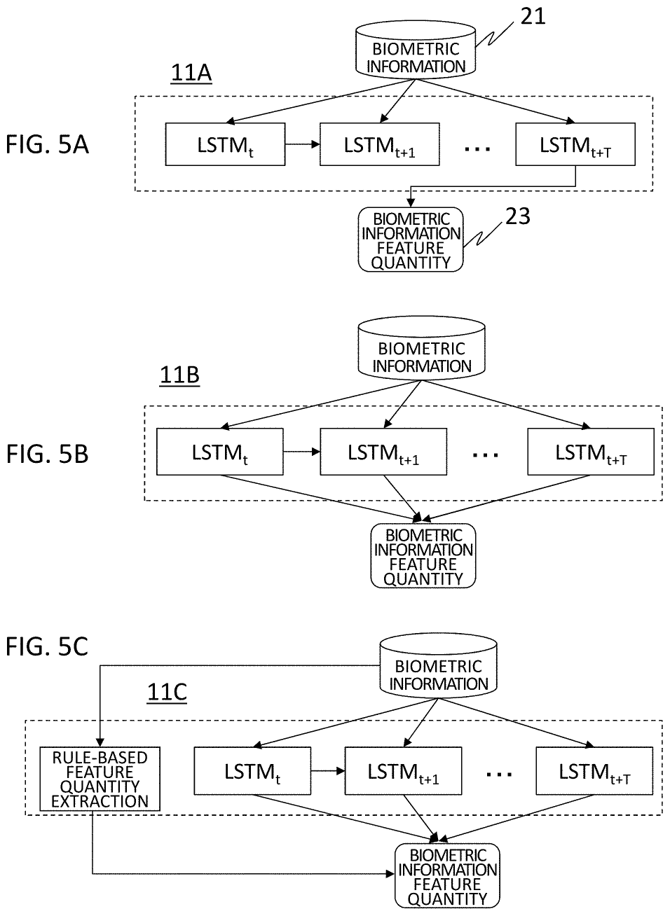

FIGS. 5A to 5C illustrate configuration examples of the biometric information analysis unit 11. In a configuration example 11A illustrated in FIG. 5A, which is a learning model including an encoder that incorporates a plurality of LSTM extractors, biometric information measured by each LSTM extractor within a fixed time interval T is read, a temporal change in the biometric information is learned, and a biometric information feature quantity is output at time t+T. A configuration example 11B illustrated in FIG. 5B outputs the biometric information feature quantity from each LSTM extractor at each time. A configuration example 11C illustrated in FIG. 5C is configured by combining a first extractor for extracting a rule-based feature quantity, for example, a statistical feature quantity such as an average value, a standard deviation, a median value, a most frequent value, a skewness, and a kurtosis within a time interval T from time-series biometric information and a second extractor configured with each LSTM extractor illustrated in FIG. 5B to output a fused feature quantity by fusing the statistical feature quantity that is the output of the first extractor and the time-series feature quantity extracted by the second extractor. In addition, although FIGS. 5A to 5C illustrate an example in which an extractor of the LSTM method is used, a recurrent neural network (RNN) method and a gated recurrent unit (GRU) method, which are known simplified versions, may be used.

[Step S42]

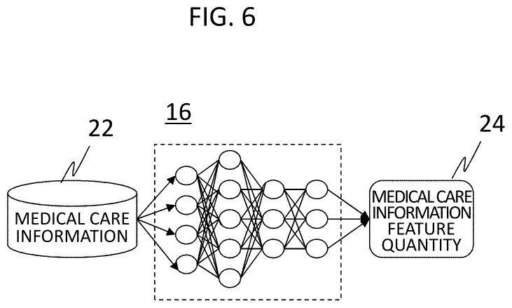

The medical care information analysis unit 16 receives medical care information 22 of the patient and extracts a medical care information feature quantity 24 by using the machine learning method. The medical care information includes age, sex, medical history, various test results, and the like of a patient, and similarly to the biometric information, data acquired in advance for a large number of patients are read and used. FIG. 6 illustrates a configuration example of the medical care information analysis unit 16. In the illustrated example, a deep neural network (DNN) method, which is a known machine learning method, is used.

[Step S43]

The feature mutation learning unit 13 receives the biometric information feature quantity 23 and the medical care information feature quantity 24 respectively extracted in steps S41 and S42, and first, the feature fusing unit 12 fuses the feature quantities to generate a fused feature quantity 25. As the method of fusing, the multi-dimensional vectors representing the feature quantity may be simply added, or the dimensions may be compressed and, after that, the addition may be performed, or the addition may be performed and, after that, the dimensions may be compressed. In addition, the feature mutation learning unit 13 executes learning of predicting the fused feature quantity 25 from only the biometric information feature quantity 23. This is processing of learning the relationship between a certain type of feature quantity and another type of feature quantity and, herein, the learning is referred to as feature quantity mutation learning.

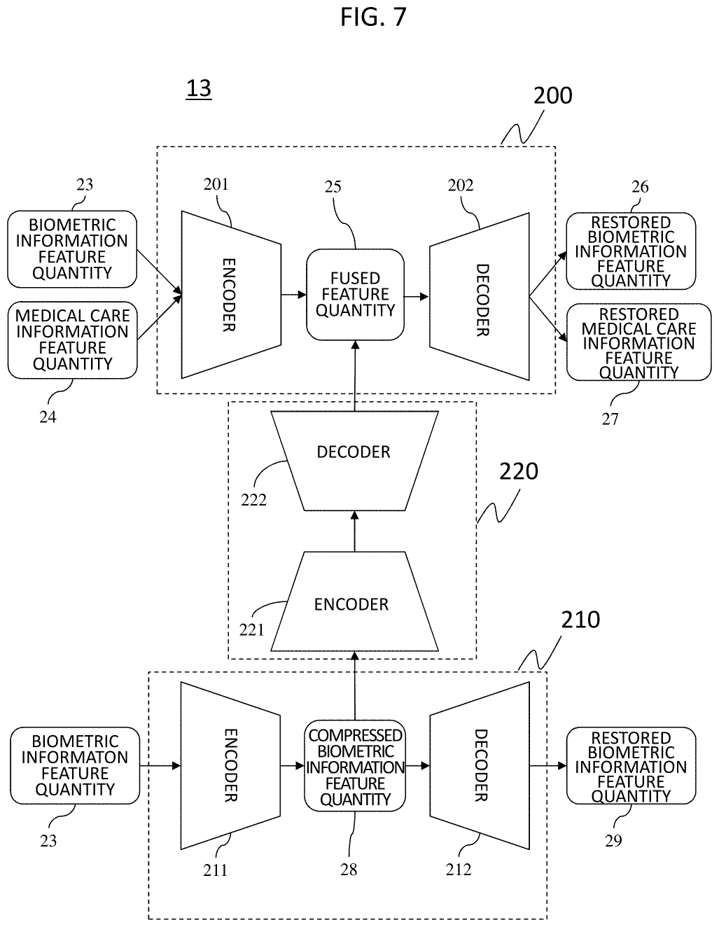

FIG. 7 illustrates a configuration example of the feature mutation learning unit 13 that realizes this feature quantity mutation function. Herein, an example is illustrated in which the feature quantity mutation learning is performed by using an auto-encoder method which is a known machine learning method. The auto-encoder method is an algorithm for dimension compression using the neural network, and uses an auto-encoder configured with an encoder and a decoder. With respect to the input data, the encoder using the neural network non-linearly compresses the dimension of the input data to generate a compressed feature quantity. The decoder restores the compressed feature quantity to an original input data by using the neural network.

The feature mutation learning unit 13 is configured by combining three such auto-encoders. A first auto-encoder 200 is a learning model for generating the fused feature quantity 25, and an encoder 201 receives and connects the biometric information feature quantity 23 and the medical care information feature quantity 24 and performs dimension compression to generate the fused feature quantity 25. In addition, a decoder 202 receives the fused feature quantity 25 and generates a restored biometric information feature quantity 26 and a restored medical care information feature quantity 27 so as to restore the biometric information feature quantity 23 and the medical care information feature quantity 24. The restoration by the decoder 202 is performed in order to check whether the auto-encoder 200 is properly learned, that is, whether the error between the input and the output is minimized.

A second auto-encoder 210 is a learning model for generating a compressed biometric information feature quantity 28. An encoder 211 receives the biometric information feature quantity 23 and performs the dimension compression, and a decoder 212 receives the compressed biometric information feature quantity 28 and generates a restored biometric information feature quantity 29 so as to restore the biometric information feature quantity 23. Restoration by the decoder 212 is also performed to check the learning effect of the auto-encoder 210.

A third auto-encoder 220 is a learning model for mutating the compressed biometric information feature quantity 28 into the fused feature quantity 25. An encoder 221 receives the compressed biometric information feature quantity 28, performs the dimension compression, and outputs the obtained further compressed feature quantity to a decoder 222. The decoder 222 performs the learning so that the compressed feature quantity can be restored to the fused feature quantity 25. Therefore, learning can be performed so that the biometric information feature quantity 23 is mutated into the fused feature quantity 25 generated by fusing the biometric information feature quantity 23 and the medical care information feature quantity 24.

In the learning process, the parameters of the neural network in the auto-encoder are estimated by iterative optimization processing. The optimization processing is performed so as to minimize a predetermined learning error. The learning may be performed by each of the three auto-encoders 200, 210, and 220 so as to minimize the error between the input and the output, or may be performed as a whole. That is, optimization can be performed so as to minimize the error between the restored biometric information feature quantity 26 and the biometric information feature quantity 23, the error between the restored medical care information feature quantity 27 and the medical care information feature quantity 24, the error between the restored biometric information feature quantity 29 and the biometric information feature quantity 23, and the error between the fused feature quantity 25 and the compressed biometric information feature quantity 28 as a whole. Furthermore, it is also possible to specify, in advance, a feature quantity that is considered to have a high contribution to the patient state prediction based on medical knowledge and the like and to apply specific weighting to minimize the error.

[Step S44]

The state prediction unit 15 generates a learning model that predicts the patient state by using the fused feature quantity 25 generated by the feature mutation learning unit 13. In the learning process, the DNN (deep neural network) method, which is a known machine learning method, may be used.

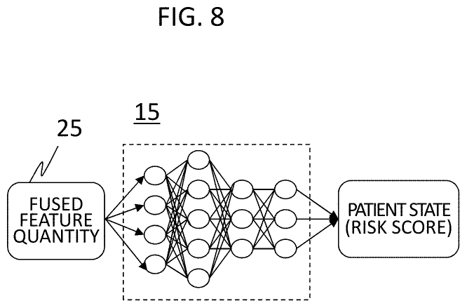

The neural network is a machine learning model configured with a plurality of artificial neurons called perceptrons. The perceptron is a decision-making model having a plurality of inputs and weighting factors corresponding to the inputs and outputs information abstracted from a prediction result or input data. The DNN is configured with a plurality of layers of the neural network. As illustrated in FIG. 8, the state prediction unit 15 uses the fused feature quantity 25 as input data, and in the optimization processing, the patient state is predicted by estimating the weighting factors of the perceptron in the neural network of each layer. The learning is performed so that the predicted value is matched with the predicted value derived from the biometric information and the patient information.

The predicted value of the patient state is a value obtained by numericalizing, for example, the outbreak risk and severity for a predetermined disease. The predicted value may be obtained by weighting and adding numerical values obtained from the medical care information and the biometric information and may be expressed as a scalar value from 0 to 100. Alternatively, the predicted value may be expressed as a discrete numerical value by dividing the risk level or severity into a plurality of steps. The predicted value may be a text representing the patient state or may be a combination of a numerical value and a text.

By the above-described steps S41 to S44, the processing of the learning function illustrated in FIG. 3A is completed. Next, as illustrated in FIG. 3B, the prediction apparatus 100A according to this embodiment performs the prediction (performs the inference) of the patient state by using the biometric information analysis unit 110, the feature mutation unit 130, and the state prediction unit 150, which are configured with the learned learning model.

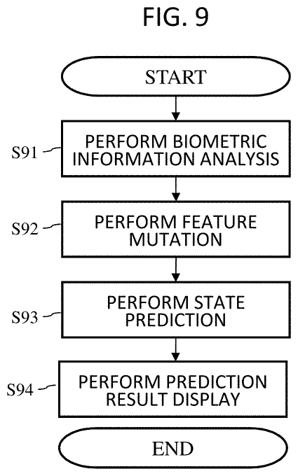

The processing of the inference function block illustrated in FIG. 3B will be described in detail below by using the flowchart of predicting the patient state illustrated in FIG. 9.

[Step S91]

The biometric information analysis unit (feature extraction unit) 110 receives biometric information 31 of the patient who is a prediction target and extracts the biometric information feature quantity 33 by using the learning parameters generated by the biometric information analysis unit 11.

[Step S92]

The feature mutation unit 130 receives biometric information feature quantity 33, predicts a fused feature quantity obtained by fusing the biometric information feature quantity and the medical care information feature quantity, and generates a fused feature quantity (predicted value) 35.

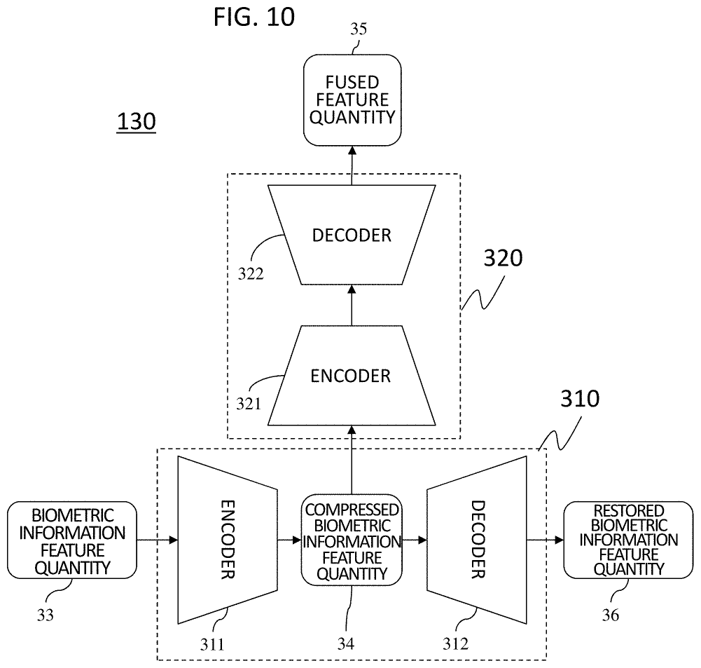

FIG. 10 illustrates a functional block diagram of the feature mutation unit 130. As illustrated in FIG. 10, the feature mutation unit 130 is configured with a learned model (auto-encoders 310 and 320) that functions as an inference processing unit among the learning models of the feature mutation learning unit 13 illustrated in FIG. 7. Each of an encoder 311 and a decoder 312 of the auto-encoder 310 is an inference processing unit corresponding to each of the encoder 211 and the decoder 212. Each of an encoder 321 and a decoder 322 of the auto-encoder 320 is an inference processing unit corresponding to each of the encoder 221 and the decoder 222.

With respect to the input of the biometric information feature quantity 33 of the prediction target patient extracted by the biometric information analysis unit 110 of FIG. 3B, the encoder 311 and the decoder 312 generate a compressed biometric information feature quantity 34 and a restored biometric information feature quantity 36 by using the parameters of the neural network learned by the encoder 211 and the decoder 212.

The encoder 321 and the decoder 322 execute the processing of mutating the compressed biometric information feature quantity 34 into the fused feature quantity 35 by using the parameters of the neural network learned by the encoder 221 and the decoder 222.

[Step S93]

The state prediction unit 150 receives the fused feature quantity 35 (predicted value), predicts the patient state by using the learned learning model, and outputs the patient state. As described in step S44 of the learning step, the patient state is output as a numericalized predicted value such as an outbreak risk score.

Furthermore, the state prediction unit 150 may estimate the uncertainty (certainty factor) of the prediction result in the estimation process. The estimation of the uncertainty of the prediction result can be performed by using a Monte Carlo dropout method, which is a known method. In the dropout method, the weight of the neural network constituting the learning model of the state prediction unit 150 can be randomly set to 0. By using the dropout method during inference, the prediction result is sampled from the distribution of the weights. By repeating such inference using the dropout method any number of times, the distribution of the prediction result can be estimated, and the uncertainty (certainty factor) of the prediction result can be estimated. Therefore, the user can make a decision regarding the patient state by referring to not only the prediction result but also the certainty factor of the prediction model regarding the prediction result.

[Step S94]

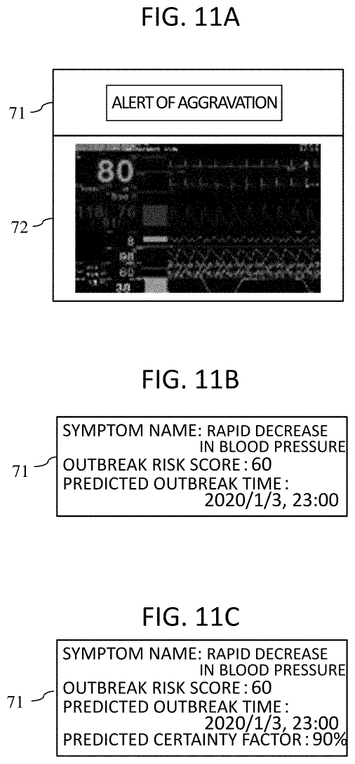

The prediction apparatus 100A allows the display control unit 9 to display the output of the state prediction unit 150 on the display 70. Although the display form is not particularly limited, a display example of the prediction result is illustrated in FIGS. 11A to 11C. FIG. 11A illustrates an example of displaying, side by side, a block 71 that displays the input patient information (biometric information or both biometric information and medical care information) used for the prediction and a block 72 that displays the prediction result of the patient state. In this example, in a case where a prediction score obtained by the state prediction unit 150 exceeds a predetermined threshold value, an alert screen is displayed in the block 71. When the user selects the content of the alert via a touch panel of the display 70 or the display control unit 9, as illustrated in FIG. 11B, the block 72 of FIG. 11A is changed to a screen of displaying specific contents of the alert such as a symptom name, a risk score, and a predicted outbreak time. In addition, in step S94 of the prediction step, in a case where the state prediction unit 150 estimates the certainty factor of the prediction result, as illustrated in FIG. 11C, the certainty factor regarding the prediction result may be displayed together.

As described above, according to the prediction method and the prediction apparatus according to this embodiment, even though the biometric information and the medical care information of which number of types is different from that of the learning data are input for the prediction model generated by using the learning data of a large number of types of the biometric information and the medical care information, it is possible to predict the patient state with high accuracy by generating a fused feature quantity that can reflect the feature quantity of a large number of types of data.

### Second Embodiment

In the first embodiment, the fused feature quantity is generated by fusing the feature quantities of different types of the patient information (biometric information and medical care information) for a large number of patients, the relationship between the feature quantity of the biometric information and the fused feature quantity is learned, the fused feature quantity is estimated from the feature quantity of the biometric information of the input prediction target patient, and the predicted value of the patient state is obtained from the estimated fused feature quantity. However, in this embodiment, the relationship between the feature quantity extracted from the large number of items of biometric information and the feature quantity extracted from the small number of items of biometric information which is a portion of the large number of items of biometric information is learned, the feature quantity of the large number of items of biometric information is estimated by inputting the small number of items of biometric information, and then, the state prediction is performed.

Namely, the prediction method according to the second embodiment includes an analysis step of extracting a feature quantity by analyzing the large number of items of biometric information of the patient and the small number of items of biometric information which is a portion of the large number of items of biometric information, a learning step of learning a relationship between the feature quantity of the large number of items of biometric information and the feature quantity of the small number of items of biometric information, a feature quantity mutation step of estimating the feature quantity of the large number of items of biometric information from the input of only the small number of items of biometric information by using the learned relationship of the feature quantity, and a prediction step of predicting a patient state by using the feature quantity obtained in the feature quantity mutation step. The biometric information obtained as time-series measurement data includes various items such as body temperature, blood pressure, pulse rate, electrocardiogram, blood oxygen concentration, and electroencephalogram. Among the biometric information, the biometric information which has as many as items as possible is the large number of items of biometric information, and the biometric information which has a small number of items and of which items that can be acquired by the facility are limited is the small number of items of biometric information. Hereinafter, the former is abbreviated as L biometric information, and the latter is abbreviated as S biometric information.

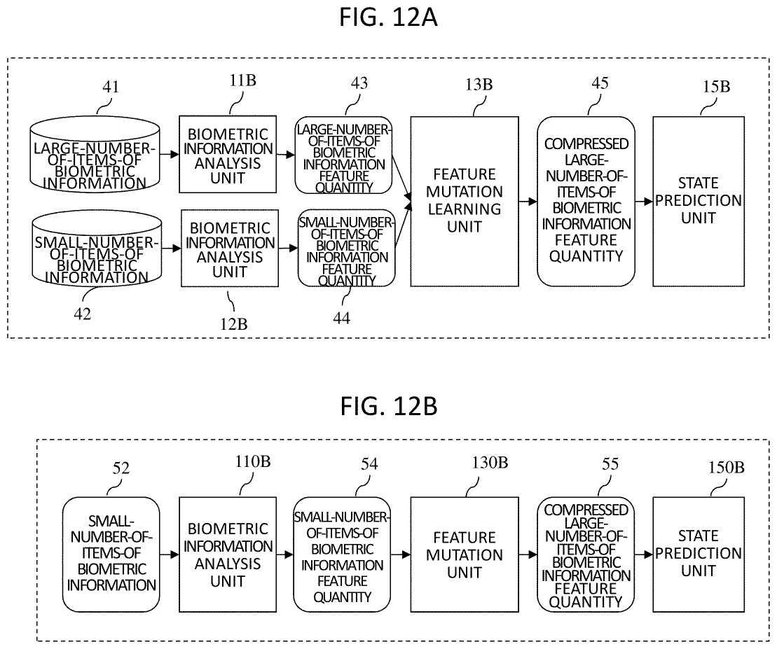

The configuration of a prediction apparatus 100B according to this embodiment is similar to the configuration of the first embodiment illustrated in FIGS. 1 and 2 and includes a feature extraction unit, a feature mutation unit, and a prediction unit, and each of the units is provided with a learning model. However, with respect to the feature extraction unit, the functional unit corresponding to the medical care information analysis unit of the first embodiment is omitted, and the processing of each unit is different. Hereinafter, the prediction method and apparatus according to this embodiment will be described with reference to FIGS. 12A and 12B. In addition, in the description of the second embodiment, the same components and processing as those in the first embodiment are denoted by the same reference numerals, and the description thereof is omitted.

FIGS. 12A and 12B are block diagrams illustrating two functions executed by the CPU 1 of the prediction apparatus 100B according to this embodiment, that is, a learning function for generating a prediction model and a prediction function using the prediction model. As the function for generating a prediction model, as illustrated in FIG. 12A, the CPU 1 is provided with first and second biometric information analysis units (feature extraction units) 11B and 12B and a feature mutation learning unit 13B, and a state prediction unit 15B. Each of the units is basically configured with a machine learning model, and by performing predetermined learning, the biometric information analysis unit (feature extraction unit) 110, the feature mutation unit 130, and the state prediction unit 150 illustrated in FIG. 12B are generated. Then, the generated biometric information analysis unit 110, the generated feature mutation unit 130, and the generated state prediction unit 150 perform the state prediction of a predetermined patient.

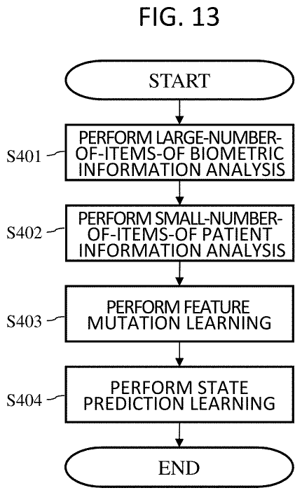

First, the processing of the learning function block illustrated in FIG. 12A will be described by using the flowchart illustrated in FIG. 13. First, in step S401, the biometric information analysis unit 11B receives an L biometric information 41, which is a large number of items of the vital data of the patient, and extracts an L biometric information feature quantity 43. In step S402, the biometric information analysis unit 12B receives a small number of types of biometric information (hereinafter, referred to as S biometric information) 42 that is a portion of the Lbiometric information 41 and extracts an S biometric information feature quantity 44. In step S403, the feature mutation learning unit 13B receives the L biometric information feature quantity 43 and the S biometric information feature quantity 44 and, first, generates a compressed L biometric information feature quantity 45 obtained by compressing the number of dimensions of the L biometric information feature quantity 43. In addition, the feature mutation learning unit 13B executes a feature quantity mutation learning function of predicting the compressed L biometric information feature quantity 45 from only the S biometric information feature quantity 44. In step S404, the state prediction unit 15B generates a learning model that predicts the patient state by using the compressed L biometric information feature quantity 45.

The configurations of the biometric information analysis units 11B and 12B are the same as the configuration of the biometric information analysis unit 11 of the first embodiment except for the difference in the amount of information to be processed and, as illustrated in FIGS. 5A to 5C, a learning model using the LSTM extractor can be adopted.

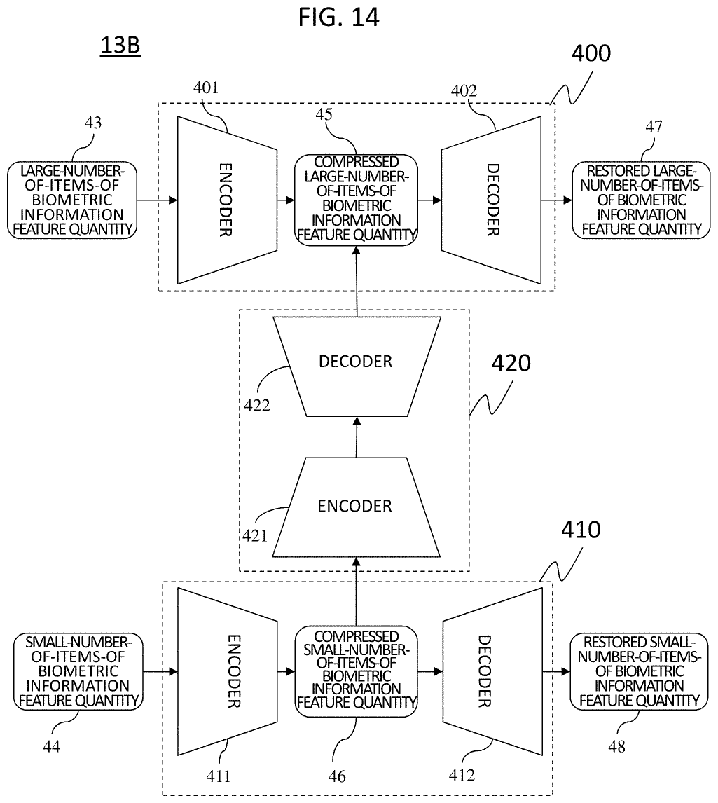

The configuration of the feature mutation learning unit 13B is similar to that of the feature mutation learning unit 13 of the first embodiment and includes three auto-encoders 400 to 420 as illustrated in FIG. 14.

An encoder 401 of the auto-encoder 400 receives a large number of items of the L biometric information feature quantity 43, performs the dimension compression, and generates the compressed L biometric information feature quantity 45. A decoder 402 receives the compressed L biometric information feature quantity 45 and generates a restored L biometric information feature quantity 47 so as to restore the L biometric information feature quantity 43.

An encoder 411 of the auto-encoder 410 receives a small number of items of the S biometric information feature quantity 44, performs the dimension compression, and generates a compressed S biometric information feature quantity 46. A decoder 412 receives the compressed S biometric information feature quantity 46 and generates a restored S biometric information feature quantity 48 so as to restore the S biometric information feature quantity 44.

The restoration of the decoders 402 and 412 is performed to check the learning effect of the learning model.

An encoder 421 of the auto-encoder 420 receives the compressed S biometric information feature quantity 46, performs the dimension compression, and outputs the further compressed feature quantity to a decoder 422. The decoder 422 performs learning so that the compressed feature quantity can be restored to a large number of items of the compressed L biometric information feature quantity 45. Therefore, it is possible to perform learning so that the S biometric information feature quantity 44 is mutated into the compressed L biometric information feature quantity 45 generated by being compressed from the L biometric information feature quantity 43.

In the optimization processing of the learning process, the optimization can be performed so as to minimize the error between the restored L biometric information feature quantity 47 and the L biometric information feature quantity 43, the error between the restored S biometric information feature quantity 48 and the S biometric information feature quantity 44, and the error between the compressed L biometric information feature quantity 45 and the compressed S biometric information feature quantity 46 as a whole. Furthermore, it is also possible to specify, in advance, a feature quantity that is considered to have a high contribution to the patient state prediction based on medical knowledge and the like and to apply specific weighting to minimize the error.

Similarly to the state prediction unit 15 of the first embodiment, as the learning model for predicting the patient state in step S404, the DNN method, which is a known machine learning method, can be used, and thus, in the optimization processing, learning is performed so that the patient state is predicted by estimating the weighting factors of the perceptron in the neural network of each layer.

By the above-described steps S401 to S404, the learning of the learning model illustrated in FIG. 12A is completed.

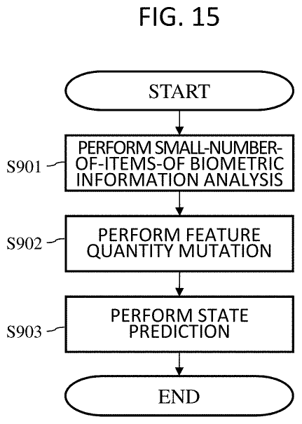

Next, a prediction function of the patient state using the learning model learned in the above-described steps will be described with reference to the flowchart of FIG. 15. First, in step S901, a biometric information analysis unit 110B receives S biometric information 52 of the patient as the prediction target and extracts an S biometric information feature quantity 54. In step S902, a feature mutation unit 130B receives the S biometric information feature quantity 54 and estimates a compressed L biometric information feature quantity 55. In step S903, a state prediction unit 150B receives the estimated compressed L biometric information feature quantity 55 and predicts the patient state by using the learned model.

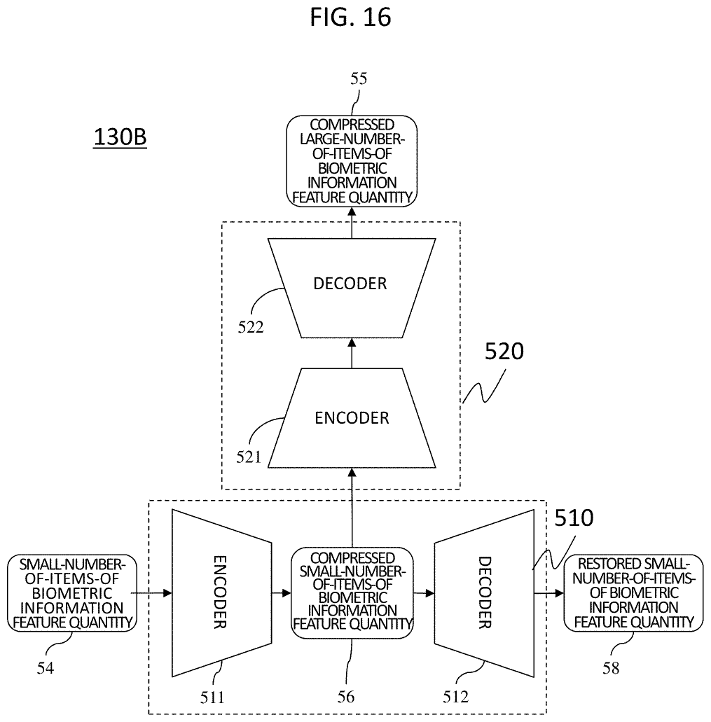

FIG. 16 illustrates a configuration example of the feature mutation unit 130B that performs the inference function. Similarly to the feature mutation unit 130 of the first embodiment, the feature mutation unit 130B of this embodiment is also configured with a learned model (auto-encoders 510 and 520) that functions as an inference processing unit among the learning models of the feature mutation learning unit 13B. Each of an encoder 511 and a decoder 512 of the auto-encoder 510 is an inference processing unit corresponding to each of the encoder 411 and the decoder 412 of FIG. 13. Each of an encoder 521 and a decoder 522 of the auto-encoder 520 is an inference processing unit corresponding to the encoder 421 and the decoder 422 of FIG. 13, respectively.

With respect to the input of the S biometric information feature quantity 54 of the prediction target patient extracted by the biometric information analysis unit 110B of FIG. 12B, the encoder 511 and the decoder 512 of the auto-encoder 510 generate a compressed S biometric information feature quantity 56 and a restored S biometric information feature quantity 58 by using the parameters of the neural network learned by the encoder 411 and the decoder 412.

The encoder 521 and the decoder 522 execute the processing of mutating the compressed S biometric information feature quantity 56 into the compressed L biometric information feature quantity 55 by using the parameters of the neural network learned by the encoder 421 and the decoder 422.

The state prediction unit 150B receives the compressed L biometric information feature quantity 55 as an input, predicts the patient state by using the learning model (DNN) learned in step S404 of FIG. 13, and outputs the patient state. The output method is the same as that of the first embodiment, and the predicted state and the biometric information used for the prediction are displayed together on the display 70. In addition, in this embodiment, it is possible to estimate the certainty factor by using the Monte Carlo dropout method at the time of inference by the state prediction unit 150B, and in this case, the certainty factor may be displayed together with the prediction result.

As described above, according to this embodiment, with respect to the prediction model generated using the learning data of the large number of items of biometric information, even though the biometric information of which number of items is different from that of the learning data or the data that lacks a portion of the biometric information due to abnormality of the biometric information measurement device or missing measurement is input, it is possible to predict the patient state with high accuracy by generating a feature quantity that can reflect the feature quantity of a large number of items of data of the biometric information.

In addition, the invention is not limited to the above-described embodiments, and various modifications are included. For example, the above-described embodiments have been described in detail for the better understanding of the invention, and thus, the above-described embodiments are not necessarily limited to those including all configurations of the description. As described above, it is needless to say that the invention is not limited to the prediction method and the prediction apparatus and can be realized as a prediction apparatus connected to a patient information recording device via a network and a prediction method thereof. In addition, a portion of the configuration of one embodiment can be replaced with the configuration of another embodiment, and the configuration of another embodiment can be added to the configuration of one embodiment. In addition, it is possible to add, delete, and/or replace other configurations with respect to a portion of the configurations of each embodiment.

Furthermore, although the example of creating the program that realizes a portion or all of the above-described each configuration, function, processing unit, and the like has been described, some or all thereof may be realized by hardware, for example, by designing with an integrated circuit.

What is claimed is:

1. A prediction apparatus comprising:
a feature extraction unit that receives biometric information of a patient as an input and extracts a feature quantity of the biometric information;
a feature mutation unit that receives the feature quantity of the biometric information extracted by the feature extraction unit as an input and outputs a predicted value of an integrated feature quantity of patient information including biometric information other than the biometric information input to the feature extraction unit; and
a state prediction unit that receives the predicted value of the integrated feature quantity as an input and outputs risk information of the patient,
wherein the feature mutation unit includes a learning model that learns a relationship between the feature quantity of the biometric information and the integrated feature quantity.

2. The prediction apparatus according to claim 1, further comprising a feature integration unit that generates the integrated feature quantity.

3. The prediction apparatus according to claim 2, wherein the integrated feature quantity generated by the feature integration unit is a fused feature quantity obtained by fusing a biometric information feature quantity and a medical care information feature quantity obtained respectively for biometric information and medical care information of a large number of patients.

4. The prediction apparatus according to claim 2, wherein the integrated feature quantity generated by the feature integration unit is a compressed feature quantity obtained by compressing a biometric information feature quantity obtained from a large number of items of biometric information of a large number of patients.

5. The prediction apparatus according to claim 2, further comprising a feature restoration unit that restores the integrated feature quantity to a feature quantity of each patient information.

6. The prediction apparatus according to claim 5,
wherein the feature integration unit and the feature restoration unit include an encoder that receives the feature quantity of each patient information as an input and outputs the integrated feature quantity and a decoder that receives the integrated feature quantity as an input and outputs a restored feature quantity of each patient information, and
wherein learning is performed so that the input feature quantity of each patient information is matched with the restored feature quantity.

7. The prediction apparatus according to claim 5, further comprising:
a compression unit that generates a compressed feature quantity by compressing the feature quantity of the biometric information extracted by the feature extraction unit; and
a restoration unit that restores the compressed feature quantity compressed by the compression unit,
wherein the feature mutation unit is learned so that the feature quantity of the biometric information input to the compression unit is matched with the feature quantity of the biometric information restored by the restoration unit and so that the feature quantity of each patient information input to the feature integration unit is matched with the feature quantity of each patient information restored by the feature restoration unit.

8. The prediction apparatus according to claim 1,
wherein the feature mutation unit includes a compression unit that generates a compressed feature quantity by compressing the feature quantity of the biometric information extracted by the feature extraction unit, and
wherein the feature mutation unit receives the compressed feature quantity generated by the compression unit as an input and outputs the integrated feature quantity.

9. The prediction apparatus according to claim 8, further comprising a restoration unit that restores the compressed feature quantity,
wherein the compression unit and the restoration unit are learned so that the feature quantity of the biometric information input to the compression unit is matched with the feature quantity restored by the restoration unit.

10. The prediction apparatus according to claim 1, wherein the feature extraction unit includes a neural network that extracts a feature quantity of time-series data.

11. The prediction apparatus according to claim 1, wherein the state prediction unit includes a neural network that is learned from the integrated feature quantity so as to output risk information of the patient.

12. The prediction apparatus according to claim 1, wherein the state prediction unit outputs the risk information of the patient as a numericalized score.

13. The prediction apparatus according to claim 1, wherein the state prediction unit calculates a certainty factor of a prediction result by a Monte Carlo dropout method.

14. The prediction apparatus according to claim 1, further comprising an output device that displays an output of the state prediction unit.

15. A prediction method comprising:
an analysis step of analyzing patient information including a large number of pieces of biometric information and extracting a feature quantity of the patient information for each type of the patient information or each item of the biometric information;
an integration step of generating an integrated feature quantity by integrating the feature quantities extracted for each type or each item;
a learning step of learning a relationship between the feature quantity for each type or each item and the integrated feature quantity;
a feature quantity mutation step of predicting a predicted value of the integrated feature quantity based on the relationship obtained in the learning step, by using a feature quantity of a specific biometric information as an input; and
a prediction step of predicting a specific state of a patient by using the predicted value of the integrated feature quantity.

16. The prediction method according to claim 15,
wherein the patient information including the biometric information includes biometric information and medical care information of the patient, and
wherein, in the integration step, a fused feature quantity is generated by fusing the feature quantity of the biometric information and a feature quantity of the medical care information.

17. The prediction method according to claim 15,
wherein the patient information including the biometric information includes a plurality of items of biometric information, and
wherein, in the integration step, a compressed feature quantity is generated by compressing the feature quantity for each item.

18. A prediction program for causing a computer to execute:
an analysis step of analyzing patient information including a large number of pieces of biometric information and extracting a feature quantity of the patient information for each type of the patient information or each item of the biometric information;
an integration step of generating an integrated feature quantity by integrating the feature quantities extracted for each type or each item;
a learning step of learning a relationship between the feature quantity for each type or each item and the integrated feature quantity;
a feature quantity mutation step of predicting a predicted value of the integrated feature quantity based on the relationship obtained in the learning step, by using a feature quantity of a specific biometric information as an input; and
a prediction step of predicting a specific state of a patient by using the predicted value of the integrated feature quantity.
